(19) United States
(12) Patent Application Publication
(10) Pub. No.: US 2010/0228086 A1
(43) Pub. Date: Sep. 9, 2010

(54) SIDE VIEWING ENDOSCOPE SYSTEM

(76) Inventors: Tomohiro OHKI, Saitama (JP); Shozo IYAMA, Saitama (JP)

Correspondence Address: BIRCH STEWART KOLASCH & BIRCH, PO BOX 747, FALLS CHURCH, VA, 22040-0747, US

(21) Appl. No.: 12/719,727

(22) Filed: Mar. 8, 2010

(30) Foreign Application Priority Data
Mar. 9, 2009 (JP) 2009-054458
Mar. 9, 2009 (JP) 2009-054459
Apr. 3, 2009 (JP) 2009-090779
Jul. 15, 2009 (JP) 2009-166360

(51) Int. Cl. A61B 1/00; A61B 10/04

(52) U.S. Cl. 600/106; 600/113

(57) ABSTRACT

A side viewing endoscope system is discloses that a mother endoscope of a side viewing type provided an endoscopic observation means is disposed on a side surface of a rigid distal end portion, a instrument rising chamber installed of a instrument riser member for rising up or sinking down of a treatment instrument in the rigid distal end portion, a biopsy channel provided through an axial direction of the insertion section being communicated with said instrument rising chamber, a daughter endoscope having a slender insertion section being inserted into the biopsy channel and having a viewing field in a forward direction of the distal end of the slender insertion section, a perforation window formed at the rigid distal end portion and having a viewing field of the forward direction of the biopsy channel, and the distal end portion being introduced through the biopsy channel at a position not to be protruded from the outer surface of the rigid distal end.

### BACKGROUND OF THE INVENTION

1. Field of the Invention

The present invention relates to a side viewing endoscope system which can ensure a front visual field when an insertion section is inserted into an insertion path in a side viewing endoscope having an observation visual field in a direction substantially crossing the axis of the insertion section at a right angle.

2. Description of the Related Art

An endoscope has an insertion section connected to a manipulation section, and the insertion section is constituted by a flexible portion disposed connecting to the manipulation section, an articular flexing portion constituting a distal end portion of the flexible portion, and a rigid distal end portion at a distal end of the articular flexing portion. At the distal end, an endoscopic observation means including an illumination portion and an observation portion is disposed and is provided with a biopsy channel through which treatment instruments such as forceps are inserted. The treatment instruction instrument is led out from the biopsy channel toward the direction of the observation field by the endoscopic observation means. One type endoscope has an observation field directed to the axial direction of the insertion section, the endoscope of this type is called as a straight viewing endoscope, while another type endoscope having an observation field toward substantially orthogonal direction to the axis of the insertion section is also utilized as a side viewing endoscope.

In the case of the side viewing endoscope, a flat portion is formed on a side face of the rigid distal end portion of the insertion section, and an illumination window and an observation window are disposed in this flat portion. A instrument rising chamber for leading out the treatment instrument is also formed in this flat portion. An outgoing end of a light guide is faced with the illumination window disposed in the side face of the rigid distal end portion, and a solid-state image pickup means is attached to the observation window. The light guide has flexibility in a bending direction, and a signal cable lead out of the solid-state image pickup means also has flexibility in the bending direction. Therefore, they are bent at approximately 90 degrees in the rigid distal end portion and extended from the insertion section to the manipulation section.

The biopsy channel is constituted by a path with flexibility from the manipulation section to the axial direction of the insertion section. In the rigid distal end portion, a space communicating with an instrument rising chamber opened in the side face is formed, and a instrument riser member is attached to this space. Therefore, this space is a treatment instrument rising space, and the biopsy channel extended from the manipulation section is opened in this treatment instrument riser chamber. The instrument riser member guides the treatment instrument inserted into the biopsy channel to the direction of the instrument rising chamber and is capable of rising operation in order to aim the distal end of the treatment instrument at a treatment portion. The operation of the instrument riser member is configured to be performed by remote control from the manipulation section, and thus, riser operating means including an operation lever or the like is provided on the manipulation section.

The side viewing endoscope provided with a standup mechanism of the treatment instrument is substantially configured as above, and the construction of this type of side viewing endoscope is disclosed in Japanese Unexamined Patent Application Publication No. 2007-136044, for example. Here, the side viewing endoscope is suitably used in a duodenoscope in general. The insertion section is inserted to the duodenum, the endoscopic observation means is arranged opposing the papilla, and examination and appropriate treatments are conducted. Treatment instruments used for the duodenoscope include a biliary cannulation tube, a metal stent and the like, and the treatment instrument is inserted into the duct tract through the papilla. In this way, by disposing a instrument riser member capable of standing up and down at the rigid distal end portion, the treatment instrument can be directed to a targeted portion smoothly and reliably.

Various treatment instruments are inserted into a side viewing endoscope, and the one configured so that a slender endoscope capable of insertion into the biliary tract is inserted therein is disclosed in Japanese Unexamined Patent Application Publication No. 11-42207. That is, a contrast medium tube is inserted into the biliary tract and a contrast medium is injected therein, and then, a slender endoscope consisting of a fiber scope is led out of the instrument rising chamber through the biopsy channel and inserted into the biliary tract so that an examination is made in the biliary tract.

When the insertion section of the endoscope is inserted to the duodenum, the portion goes through the stomach from the esophagus, but this insertion path has a considerable length. Moreover, the insertion path is bent in a complicated way and has an uneven structure having a constrictive portion and an expanded portion in the middle of the path. Thus, an insertion operation of the insertion section should be conducted carefully while its insertion direction is checked. In the side viewing endoscope, since the observation visual field is oriented to the direction orthogonal to the axis of the insertion section, the front in the insertion direction cannot be contained in the visual field by the endoscopic observation means. Since the endoscope has an observation visual field on the side, the front view of the distal end portion of the insertion section can be grasped to some degree, but the front view cannot be directly checked. Therefore, there is some difficulty in an operation to insert the insertion section, the insertion operation requires skills, and it is likely that the insertion operation takes time. In short, the side viewing endoscope has problems to be solved in a point of insertion operability.

### SUMMARY OF THE INVENTION

The present invention was made in view of the above problems and has an object to provide a side viewing endoscope system with a simple construction that can ensure a front visual field in the insertion direction during an insertion operation.

The present invention has another object to provide a side viewing endoscope system that, in insertion of an endoscope, the front visual field is ensured through the treatment instrument insertion channel and after the insertion section is arranged at a predetermined position, the treatment instrument insertion channel is made to exert its original function.

According to the present invention, in order to solve the above-stated objectives, there is provided a side viewing endoscope system comprising: a mother endoscope of a side viewing type in which an endoscopic observation means having an illumination portion and an observation portion is disposed on a side surface of a rigid distal end portion of an insertion section, a instrument rising chamber installed of a instrument riser member for rising up or sinking down of a treatment instrument is formed in said rigid distal end portion, a biopsy channel provided through an axial direction of said insertion section being communicated with said instrument rising chamber; a daughter endoscope having a slender insertion section being inserted into said biopsy channel of said mother endoscope and having a viewing field in a forward direction of said distal end of said slender insertion section; a perforation window formed at said rigid distal end portion of said mother endoscope and having a viewing field toward the forward direction of said biopsy channel; and said distal end portion of said daughter endoscope being introduced through said biopsy channel at a position to face said perforation window but not to be protruded from the outer surface of said rigid distal end of said mother endoscope.

As described in the above-mentioned Japanese Unexamined Patent Application Publication No. 11-42207, irrespective of an optical endoscope or an electronic endoscope, it comes into wide use that an endoscope has an insertion section which can be inserted into a biopsy channel of a side viewing endoscope. In the present invention, the endoscope of the type having a side viewing is constituted as a mother endoscope, and a daughter endoscope of a straight view having a slender insertion section is adapted to insert into the biopsy channel of the mother endoscope. By so constructing, the mother endoscope is available an observation field for the advanced route of a body cavity by means of the slender insertion section of the daughter endoscope which is inserted into the biopsy channel of the mother endoscope.

The instrument rising chamber is constituted by a recess portion opened to a side face portion of the rigid distal end portion and has a treatment instrument base attached inside. Also, at a front position of the biopsy channel in the instrument rising chamber, a front end wall is located. Even if the daughter endoscope is introduced into the instrument rising chamber through the biopsy channel as an insertion path of the daughter endoscope, the front visual field cannot be ensured in that state. Thus, a perforation window is formed in this front end wall. At least a portion on the distal end side of the rigid distal end portion can be configured by a transparent member, but the rigid distal end portion is usually configured by a non-transparent member, and in this case, the perforation window is formed by forming a through hole in the front end wall of the rigid distal end portion.

In the instrument rising chamber, the instrument riser member is arranged between the front terminal position of the biopsy channel and the distal end portion of the rigid distal end portion in which the perforation window is formed. Since the instrument riser member is to change the direction of the treatment instrument inserted into the biopsy channel to be directed to the instrument rising chamber, the slender insertion section of the daughter endoscope cannot be advanced further as it is to the perforation window in a general side viewing endoscope. Thus, in order that the slender insertion section can pass through the position of the instrument riser member, a daughter endoscope passing mechanism is disposed on the instrument riser member.

The instrument riser member is adapted to change a direction of a treatment instrument, and the treatment instrument is not inserted before going the insertion section of the mother endoscope on the area to be examined or observed such as duodenum. The treatment instrument is inserted only when the examination or observation is performed. At this time, the front visual field is not needed. Thus, the daughter endoscope passing mechanism provided on the instrument riser member can be so constituted that the instrument riser member is retreated from the treatment insertion path and the front of the biopsy channel is opened. That is, the instrument riser member has an operation range of a sunken down position to a minimum angle and risen up position at a maximum angle, further the instrument riser member can be forcedly displaced to a position beyond the minimum angle.

The instrument riser member is normally connected to a rotational shaft, and the rotational shaft is supported by a wall portion of the rigid distal end portion. The instrument riser member is configured to perform an operation by rotating the instrument riser member around the rotational shaft. Even in the most sunken down state, that is, at the minimum angular position of the instrument riser member, a guide surface thereof can be led out from the instrument rising chamber. On the other hand, the most risen up state, that is, at the maximum angular position, the instrument rising chamber is partially covered by the instrument riser member. Specifically, in a case where a direction orthogonal to the axis of the insertion section is defined as 90 degrees, generally a treatment instrument can be bent from an angle less than 90 degree (inclined toward fore direction) to an angle more than 90 degree (inclined toward rear direction).

An operation of the instrument riser member is performed on the manipulation section side. Therefore, on the manipulation section, a rising member operation means such as an operation lever or an operation knob is provided. Or otherwise a slide lever or the like can be constituted as the rising member operation means. If the operation lever or operation knob is to be used, it can be made as an independent construction for operation of the riser member, and since an operating member constituting bending operation means for the insertion section is attached to the manipulation section, the lever or knob may be disposed coaxially with the rotational shaft of the operating member. By configuring such that the bending operating means and the rising member operation means are disposed coaxially, the operating mechanism can be simplified. Transmission member including an operating wire is connected to the rising member operation means, and the transmission means is extended to the distal end of the insertion section. The operating wire can be directly connected to the instrument riser member, but it may also be so configured that the lever is connected to the rotational shaft and the transmission member is connected to the lever, by which the instrument riser member can be risen up and can be operated smoothly with a slight load.

The rising member operation means is configured capable of switching of operation modes. That is, the instrument riser member is made rotatable from the minimum angular position to the maximum angular position in an operation range of the riser member, which is a rising operation mode. The instrument riser member is operated to the retreated position by being set at an angular state smaller than the minimum angular position. This is an ex-boundary operation mode. The operation mode is configured capable of switching to the rising operation mode and the ex-boundary operation mode. In a usual state, an operation range limiting portion is provided for limiting the range within an operation range to the rising operation mode, and the operation mode can be made change-over to cancel the limitation to the above-mentioned operation range limiting portion.

The daughter endoscope passing mechanism disposed on the instrument riser member is configured by a daughter endoscope passage formed on a guide surface of the instrument riser member. That is, a through hole or a guide groove through which the slender insertion section of the daughter endoscope can be inserted is disposed in the instrument riser member. The through hole or guide groove is formed is placed at an extended position of the biopsy channel. The daughter endoscope passage can be kept usually open or otherwise may provide a shutter to be opened and closed by operation. If the daughter endoscope passage is usually kept open, the slender insertion section is passed through the daughter endoscope passage when the slender insertion section of the daughter scope is introduced therein, while a treatment instrument can be guided toward the instrument rising chamber along the guide surface of the instrument riser member when the treatment instruction is introduced therein. For that purpose, the daughter endoscope passage should have a diameter smaller than an outer diameter dimension of a usual treatment instrument to be inserted in a side viewing endoscope and also should have a diameter that the slender insertion section can be passed through.

A through hole is formed in the front end wall of the rigid distal end portion of the insertion section of the mother endoscope as a perforation window. By the above-mentioned construction, a slender insertion section of the daughter endoscope should be configured not to pass through the through hole or at least not to protrude from the rigid distal end portion. Thus, a transparent plate can be attached to the through hole for the sake of a stopper. As a result, if the slender insertion section of the daughter endoscope is inserted through the biopsy channel and when the distal end of the slender insertion section goes to a position in contact with the perforation window, the distal end is stopped at the position of the perforation window so that the slender insertion section does not protrude from the rigid distal end portion.

By setting a hole diameter of the through hole smaller than the outer diameter of the slender insertion section with the smallest diameter of a normal daughter endoscope, it can function as a restriction portion such that the slender insertion section does not pass through the through hole nor protrude from the rigid distal end portion. For example, the through hole can be made to have one or more step and its most reduced diameter has a function of the restriction portion. If the slender insertion section is smaller than the diameter of the through hole, a medical tape may be wound around an outer circumference of the distal end portion of the slender insertion section so as to increase the outer diameter thereof so that the slender insertion section is prevented not to pass through the through hole. The through hole may have a uniform diameter but can be also constituted as a tapered hole whose diameter is continuously reducing toward the front side. In this case, the tapered hole has a introductory function to be insertion the daughter endoscope, and also has a centering function of the slender insertion section with respect to the through hole.

The daughter endoscope may be an optical endoscope, or otherwise the daughter endoscope may be constituted by an electronic endoscope when the mother endoscope is an electronic endoscope. During a course of the operation that the mother endoscope is inserted into the body cavity, observation is not necessary of the visual field of the mother endoscope directed to substantially orthogonal of the axis of the insertion section, and the front visual field by the daughter endoscope is essentially necessary for the sake of insertion of the mother endoscope. A straight-view observation image can be attained by the observation portion of the daughter endoscope conjugated in the mother endoscope by displaying on a monitor screen. When the daughter endoscope is separated from the mother endoscope, the biopsy channel can leave open for inserting a treatment instrument, also the monitor screen is shifted to display a side-view observation image by the mother endoscope automatically or by a manual operation.

As mentioned above, by inserting the slender endoscope having the straight viewing into the biopsy channel, a forward field of vision can be attained with a simple construction through a perforation window disposed at the rigid distal end portion of the mother endoscope. Moreover, the biopsy channel is used as its original purpose, that is, when a treatment instrument is to be inserted, there is no need to ensure the forward visual field, therefore, the daughter endoscope should be removed beforehand from the mother endoscope.

### BRIEF DESCRIPTION OF THE DRAWINGS

Embodiments of the present invention will be described referring to the attached drawings. The present invention is not to be interpreted with limitation to the embodiments illustrated below.

FIG. 1 is a construction explanatory diagram illustrating a mother endoscope and a daughter endoscope.

FIG. 2 is an appearance view of a distal end portion in an insertion section of the mother endoscope illustrating a first embodiment of the present invention.

FIG. 3 is a longitudinal sectional view of FIG. 2.

FIG. 4 is an X-X sectional view of FIG. 3.

FIG. 5 is a construction explanatory diagram illustrating a construction of an operating mechanism of a instrument riser member.

FIG. 6 is a sectional view of riser operating means illustrated with a bending operation means.

FIG. 7 is a sectional view of a distal end portion of an insertion section of a daughter endoscope.

FIG. 8 is an explanatory diagram illustrating a construction of the mother endoscope and the daughter endoscope as electronic endoscopes in which images by the both endoscopes are switched in display.

FIG. 9 is a sectional view similar to FIG. 3 illustrating a state in which the daughter endoscope is incorporated in the mother endoscope.

FIG. 10 is a sectional view of a state in which the daughter endoscope is incorporated in the mother endoscope, illustrating a second embodiment of the present invention.

FIG. 11 is a front view of a instrument riser member in FIG. 10 in a state in which the daughter endoscope and a treatment instrument are inserted.

FIG. 12 is a construction explanatory diagram illustrating a variation of the second embodiment of the present invention in which a light source of the daughter endoscope is disposed on the mother endoscope side.

FIG. 13 is a sectional view similar to FIG. 10 illustrating a variation of the daughter endoscope in the second embodiment of the present invention.

FIG. 14 is a sectional view of an essential part illustrating a variation of a regulation portion to prevent an insertion section of the daughter endoscope from protruding from the rigid distal end portion.

FIG. 15 is a front view of FIG. 14.

FIG. 16 is a sectional view of an essential part illustrating another variation of the regulation portion to prevent the insertion section of the daughter endoscope from protruding from the rigid distal end portion.

### DESCRIPTION OF THE PREFERRED EMBODIMENTS

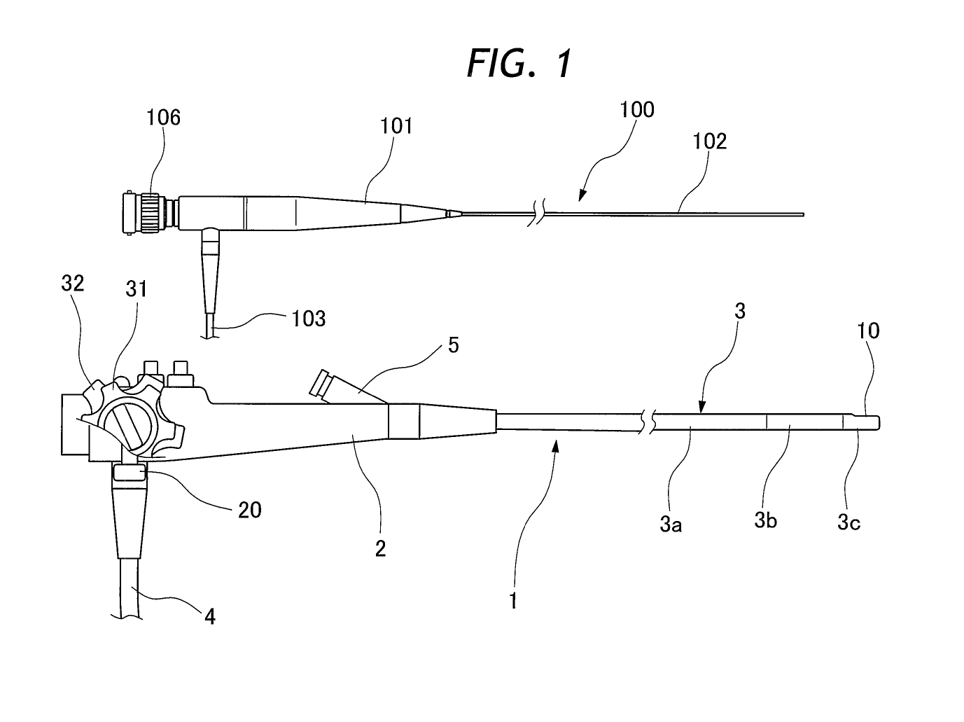

Embodiments of the present invention will be described below with reference to the attached drawings. First, FIG. 1 shows appearances of a side viewing endoscope and a slender straight viewing endoscope inserted through a biopsy channel of the side viewing endoscope. Here, in the following explanation, the side viewing endoscope is referred to as a mother endoscope 1, while the slender straight viewing endoscope to be used in conjugation of the mother endoscope 1 is referred to as a daughter endoscope 100.

In the mother endoscope 1, an insertion section 3 is connected with a manipulation section 2, and a universal cord 4 is also connected to the manipulation section 2. The insertion section 3 has a flexible portion 3a whose most part from a connection part with the manipulation section 2 is adapted to bend in an arbitrary direction along an insertion passage of the endoscope, a distal end portion of the flexible portion 3a is a articular flexing portion 3b, and a rigid distal end portion 3c is consecutively provided at a distal end position of the articular flexing portion 3b.

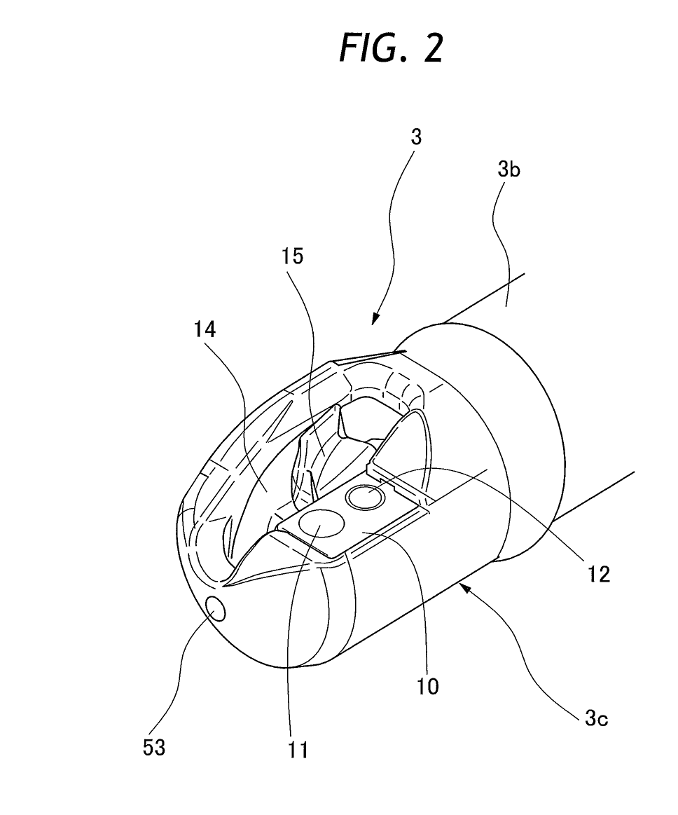

FIG. 2 shows an appearance of the distal end portion in the insertion section 3 of the mother endoscope 1. The rigid distal end portion 3c which is connected consecutively to the articular flexing portion 3b has a flat face portion 10 is formed in the side surface, and an illumination portion 11 and an observation portion 12 are provided as a endoscope observing mean in this flat face portion 10. An illumination lens is attached to the illumination portion 11, and an end face of a light guide is placed opposed to the illumination lens. Also, an objective optical system is disposed in the observation portion 12, and solid-state image pickup means is provided at an image forming position of the objective optical system. Since specific constructions of the illumination portion 11 and the observation portion 12 are known, illustration and detailed description will be omitted.

Here, a side viewing endoscope as the mother endoscope 1 is used mainly as a duodenosocpe, and this type of endoscope is inserted through a mouth to a duodenum through an esophagus and a stomach. In a cavity wall portion of the duodenum, a papilla communicating with a biliary tract is opened. Therefore, a treatment instrument such as a cannulation tube, a stent and the like is inserted through the papilla into the biliary tract or through the biliary tract into a common bile duct or the like so that predetermined examinations, treatments and the like are performed. For that purpose, an insertion path for the above-mentioned treatment instrument is disposed in the side viewing endoscope.

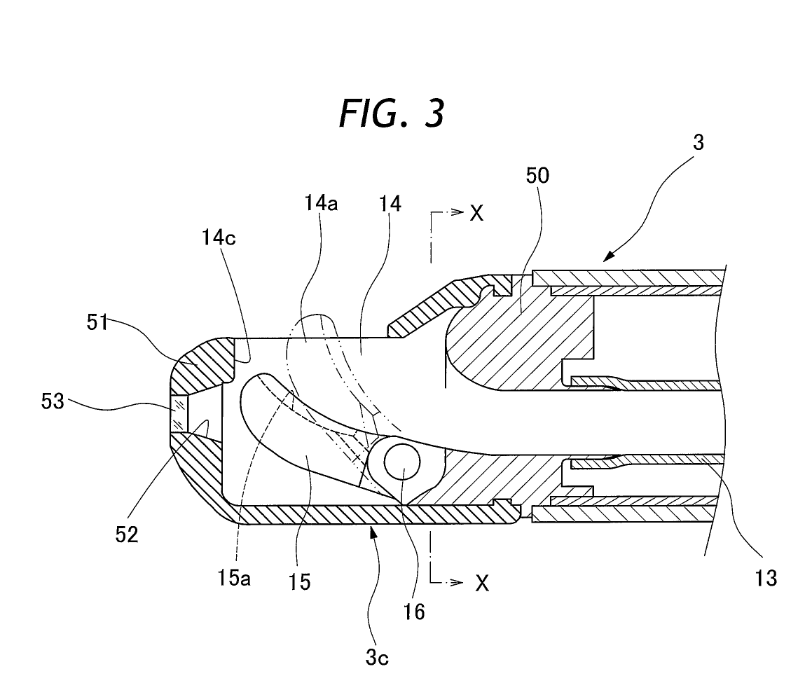

As an inlet portion in the insertion path for the treatment instrument, as shown in FIG. 1, a treatment instrument introduction portion 5 is disposed on the manipulation section 2. A construction of the rigid distal end portion 3c in the insertion section 3 is shown in FIG. 3. In this figure, reference numeral 13 denotes a biopsy channel constituted by a flexible tube, and this biopsy channel 13 is extended form the manipulation section 2 to the insertion section 3. The biopsy channel 13 is extended in the axial direction in the insertion section 3 and connected to a recess part formed in the rigid distal end portion 3c. This recess part is a instrument rising chamber 14, and the instrument rising chamber 14 is opened at a portion of the flat portion 11 as an instrument rising opening 14a. The instrument rising chamber 14 is a space for the treatment instrument introduced in the axial direction of the insertion section 3 by the biopsy channel 13 to be oriented toward a direction to be led out of the instrument rising opening 14a, and an instrument riser member 15 is attached in this instrument rising chamber 14.

The instrument riser member 15 has a construction capable of controlling an angle of the treatment instrument introduced through the biopsy channel 13 into the instrument rising chamber 14 when it is led out of the instrument rising opening 14a. The instrument rising chamber 14 is longitudinal space in the axial direction of the insertion section 2 and has a construction capable of controlling the direction to be protruded of the treatment instrument by being elongated toward the axial direction of the instrument rising opening 14a. The instrument riser member 15 has a guide surface 15a for sliding the treatment instrument and is operated to rise up or sink down by remote control from the manipulation section 2 side. In FIG. 3, a position indicated by a solid line is a minimum angular position where the instrument riser member 15 is sunken down the most, and the treatment instrument is led out to diagonally front at this time. Also, a position indicated by a dot-chain line is a maximum angular position where the instrument riser member 15 is risen up to maximum degree, and the treatment instrument is led out to inclined rear-ward direction. A moving range between the minimum angular position and the maximum angular position is an operation range of the instrument riser member 15 as a rising operation mode. In this way, by inclination and displacement between the minimum angular position and the maximum angular position by the rising operation of the instrument riser member 15, the lead-out direction of the treatment instrument is guided along the guide surface 15a of the instrument riser member 15 is controlled.

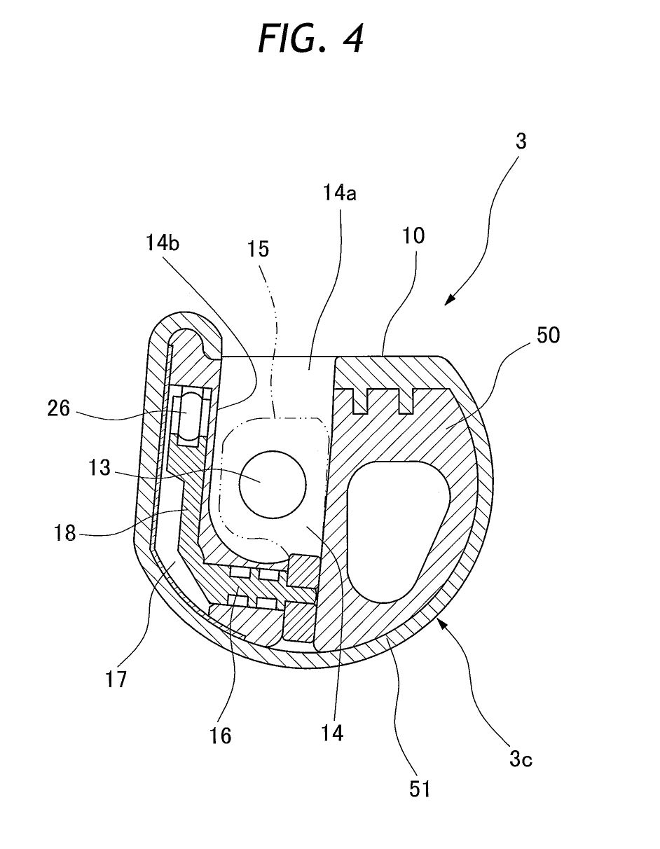

The instrument riser member 15 is, as shown in FIG. 4, rotatably supported by side wall portion 14b of the instrument rising chamber 14 of the rigid distal end portion 3 rotatably through a rotational shaft 16. The rotational shaft 16 is extended into a lever mounting space 17 and is connected to a driven lever 18 in the lever mounting space 17, and by rotational movement of the driven lever in the fore and rear direction, the instrument riser member 15 is rotationally moved around the rotational shaft 16.

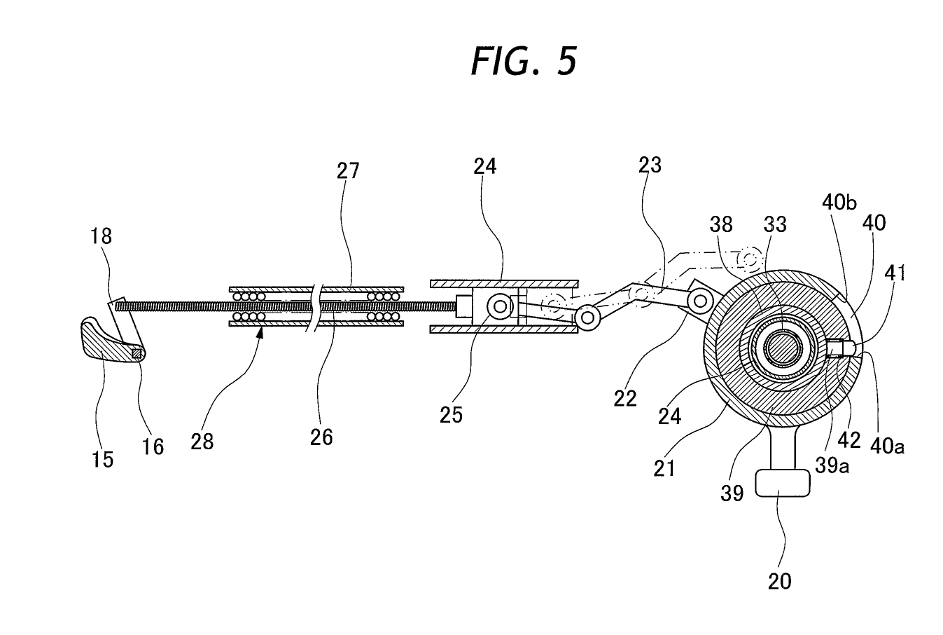

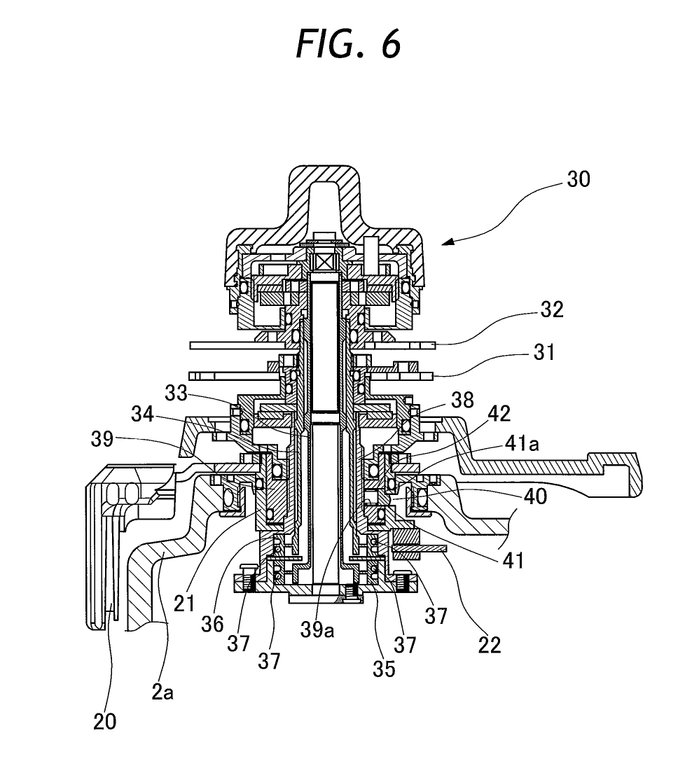

A construction of the rising member operation means for the risen up or sinking down operation of the instrument riser member 15 by remote control is shown in FIGS. 5 and 6. A riser operation lever 20 is disposed on the manipulation section 2, and this riser operation lever 20 is, as shown in FIG. 5, connected to a rotating drum 21 provided coaxially with hollow rotational shafts 33, 34 of a bending operation means 30, which will be described later. A connecting plate 22 is provided to connect with the rotating drum 21 at one end and is integrated with the riser operation lever 20. The other end of the connecting plate 22 is pivotally connected to one end of a crank member 23, while the other end of the crank member 23 being pivotally supported by a slider 25 which is slidably disposed on a slide guide 24. An operating wire 26 is connected to the slider 25, and the operating wire 26 is inserted through a flexible sleeve 27. The operating wire 26 and the flexible sleeve 27 constitute a control cable 28. The flexible sleeve 27 can be constituted by an intimately wound coil shaft, and the coil shaft is preferably wrapped with a tube made of heat-shrinkable resin.

As shown in FIG. 6, the bending operation means 30 is disposed on the manipulation section 2 for a bending operation of the articular flexing portion 3b of the insertion section 3 via remote control, the bending operation means 30 has operating knobs 31, 32 and the one operating knob 31 performs a bending operation of the articular flexing portion 3b vertically, for example, while the other operating knob 32 performs the bending operation horizontally. Coaxially disposed hollow rotational shafts 33, 34 are respectively connected to the operating knobs 31, 32. The hollow rotational shafts 33, 34 are placed in a casing 2a of the manipulation section 2 and are connected to pulleys 35, 36. A pair of operating wires 37 is wound in the pulley 35, 36, respectively. By manipulating the operating knobs 31, 32, the pulleys 35, 36 are rotated to pull into the one operating wire 37, push out of the other operating wire 37, as a result, the articular flexing portion 3b is bent along the operating wire 37 on the pulled side.

The rotating drum 21 of the riser operation lever 20 is disposed coaxially with the hollow rotational shafts 31, 32 which constitute the bending operation means 30, and a fixing shaft 38 is interposed between the rotating drum 21 and the hollow rotational shaft 32. A restricting drum 39 is connected to the fixing shaft 38 by way of screwing engagement at the position between the restricting drum 39 and the rotating drum 21, a mechanism for restricting a rotational angle of the rotating drum 21, and a rising angle of the instrument riser member 15 by the operation of the riser operation lever 20 is limited from the minimum angular position to the maximum angular position.

An arc groove 40 is formed in the rotating drum 21, while a stopper member 41 is disposed on the restricting drum 39, and the stopper member 41 is engaged with the arc groove 40. Therefore, by operating the riser operation lever 20 so as to rotate the rotating drum 21, it is operated between contact positions with groove end portions 40a, 40b on both sides of the arc groove 40. If the rotating drum 21 is rotated to the position where the stopper member 41 is brought into contact with the groove end portion 40a of the arc groove 40, the instrument riser member 15 becomes at the minimum angular position. While it is brought into contact with the groove end portion 40b, the riser operation is performed so that the instrument riser member 15 is located at the maximum angular position. This is an operation range by the rising operation mode.

The rigid distal end portion 3c has a distal end portion main body 50 made of a rigid member, and an outer peripheral portion of the distal end portion main body 50 is covered by a cover member 51. In wall faces constituting the instrument rising chamber 13, a perforation area is formed in a front end wall 14c (See FIG. 3) located in front of the instrument riser member 15. This perforation area is disposed in the axial direction of the insertion section 3 and is constituted by a through hole 52 formed in the cover member 51 and a perforation window 53 is attached to the through hole 52. The perforation window 53 is constituted by attaching a transparent plate to a through hole formed in the cover member 51.

Within the operation range by the rising operation mode of the instrument riser member 15, even at the minimum angular position, the instrument riser member 15 is interposed between the biopsy channel 13 and the perforation window 53. Then, the instrument riser member 15 is retreated from the position between the biopsy channel 13 and perforation window 53 so that the forward direction of the biopsy channel 13 can be opened. This constitutes the daughter endoscope passing mechanism and opens a space at the extended area of the biopsy channel 13 up to the perforation window 53.

The riser operation lever 20 can reset the limitation on the operation range of the rising operation mode within the both ends of the arc groove 40 of the rotating drum 21 to bring the instrument riser member 15 to the position of ex-boundary operation mode with which the instrument riser member 15 is sunken down to an angle smaller than the minimum angular position. The stopper member 41 is not fixedly disposed on the restricting drum 30 but fitted in the through hole 39a formed in the restricting drum 39 so as to retracted in and protruded out of the outer periphery of the restricting drum 39. Further, a spring 42 is mounted to urge the fixing shaft 38 to protrude in the groove 40. The stopper member 41 is formed a shoulder portion 41a which is brought into contact with an end portion of the arc groove 40 of the restricting drum 39 so as to prevent that the stopper member 41 extracts from the arc groove 40 by the function of spring 42.

By forcedly rotating the riser operation lever toward the direction where the instrument riser member 15 is brought to a further smaller angle at the time of the stopper member 41 being in contact with the groove end portion 40a of the arc groove 40 in the rotating drum 21, the stopper member 41 can be pressed down against an urging force of the spring 42, thereby restriction on the rotating drum 21 by the stopper member 41 being cancelled. For this purpose, a distal end portion of the stopper member 41 is formed as a spherical shape so that the stopper member 41 can retract to a position lower than the arc groove 40 in the rotating drum 21. As a result, the instrument riser member 15 is displaced at an angle smaller than the minimum angular position.

As mentioned above, by releasing the restriction on the operation range of the rotating drum 21 by the stopper member 41, the mode is brought into the ex-boundary operation mode, and a position of the instrument riser member 15 at this time is the retreated position. If the instrument riser member 15 is displaced to this retreated position, the forward position of the biopsy channel 13 is opened. Also, by returning the rising operation lever 20 within the range of the rising operation mode, the stopper member 41 is returned to the state engaged with the arc groove 40 by an action of the spring 42.

As mentioned above, the instrument riser member 15 can be displaced to the retreated position, and the perforation window 53 constituted by the through hole 52 is formed at an extended position of the biopsy channel 13, thereby ensuring the forward the front visual field of the insertion direction in the side-view mother endoscope 1 when the mother endoscope 1 is to be inserted into the body cavity.

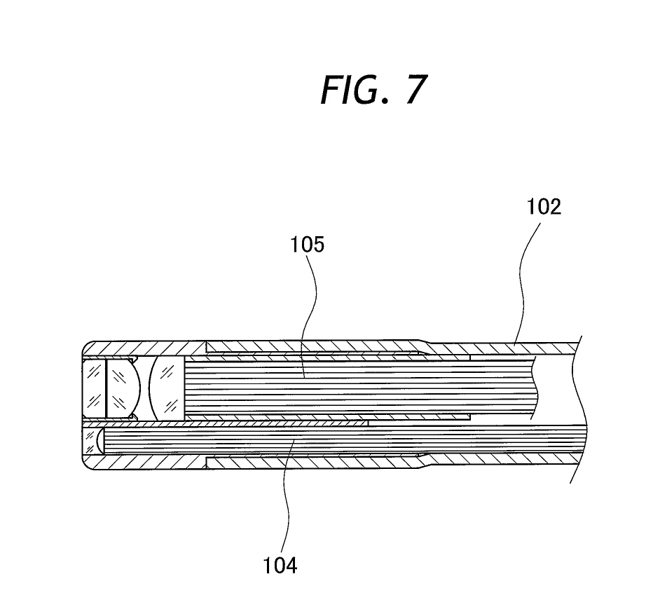

The daughter endoscope 100 is used in order to ensure the forward visual field in the insertion of the mother endoscope 1. The daughter endoscope 100 has a slender insertion section 102 connected to a manipulation section 101 similar to the mother endoscope 1, and a universal cord 103 is connected to the manipulation section 101. Here, the daughter endoscope 100 is, as shown in FIG. 7, constituted by an optical endoscope which has a light guide 104 and an image guide 105 in the slender insertion section 102. An ocular portion 106 is disposed at a rear end portion of the manipulation section 101. Although a biopsy channel or a bending operating mechanism is not disposed for the sake of reduction of the diameter of the slender insertion section 102 of the daughter endoscope 100, but those mechanisms may also be provided. In addition, the daughter endoscope 100 can be constituted such that a solid-state image pickup element is attached at an image forming position of the optical system, an image signal from the solid-state image pickup element is transmitted and applied with predetermined signal processing and can be displayed on a monitor screen.

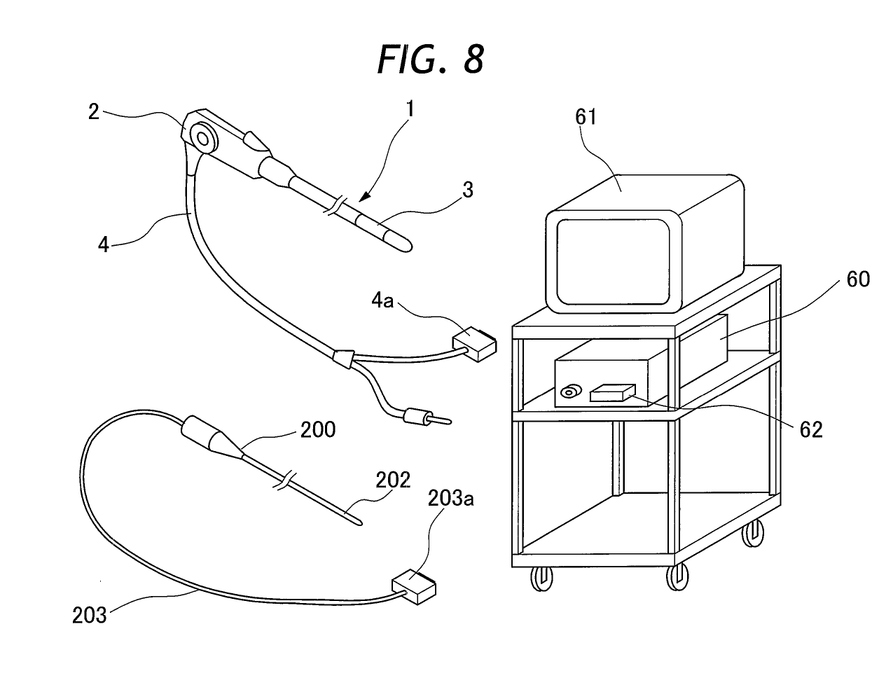

Particularly, as shown in FIG. 8, when a daughter endoscope 200 is constituted as an electronic endoscope, a connector 203a is disposed at an end portion of its universal cord 203. The observation image of the mother endoscope 1 and the observation image of the daughter endoscope 200 can be selectively displayed on a monitor 61 of a processor 60. For that purpose, a construction can be adopted to be connected with an electric connection portion 62 of the processor 60 either an electric connector 4a disposed at an end portion of the universal cord 4 of the mother endoscope 1 or the connector 203a disposed at the end portion of the universal cord 203 of the daughter endoscope 200. By so constructing, both of a side-view image from the mother endoscope 1 and a straight-view image picked up by the daughter endoscope 200 can be displayed on the monitor 61. Therefore, when the connector 4a of the mother endoscope 1 is connected to the connection portion 62, the side-view image by the mother endoscope 1 is displayed on the monitor 61, while when the connector 103a of the daughter endoscope 200 is connected to the connection portion 62, the straight-view image by the daughter endoscope 200 is displayed on the monitor 61.

In the course of operation that the mother endoscope 1 is inserted into the body cavity, the slender insertion section 102 of the daughter endoscope 100 (or the slender insertion section 202 of the daughter endoscope 200) is inserted into the biopsy channel 5 from the manipulation section 2. At the same time, the limitation on the operation range between the stopper member 41 of the restricting drum 39 and the arc groove 40 in the rotating drum 21 is released so as to bring the rising operation lever 200 into the ex-boundary operation mode. As a result, the operating wire 26 in the control cable 28 is pushed out of the flexible sleeve 27. As a result, the instrument riser member 15 is displaced to the retreated position, the daughter endoscope passing mechanism is brought into the operating state, and the forward position of the biopsy channel 13 is opened.

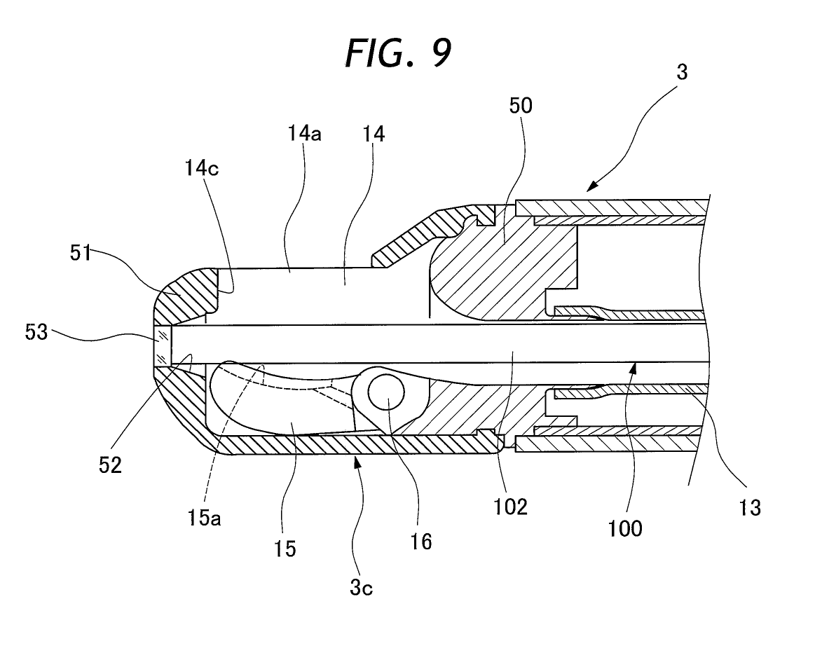

The slender insertion section 102 of the daughter endoscope 100 is introduced from the biopsy channel 13 into the instrument rising chamber 14. Since the through hole 52 is formed in the front end wall 14c of the instrument rising chamber 14, the slender insertion section 102 is, as shown in FIG. 9, inserted into the through hole 52. Even if the instrument riser member 15 does not fully open the through hole 52, in the ex-boundary operation mode, the hole is pressed by the end of the slender insertion section 102 so as to retreat from the portion of the through hole 52. The through hole 52 has a tapered face expanded toward a proximal side, thus ensuring smooth insertion of the slender insertion section 102 into the through hole 52. The perforation window 53 is disposed in the cover member 51 in the rigid distal end portion 3c, and the perforation window 53 exhibits the function as the restriction portion to prevent the distal end of the slender insertion section 102 protruding from the outer surface of the rigid distal end portion 3c, thereby the slender insertion section 102 being stopped at the position to be brought into contact with the perforation window 53, and not protrude from the rigid distal end portion 3c. Therefore, the forward visual field can be ensured by the endoscope 100 during insertion of the mother endoscope 1 into a body cavity, thus ensuring the operation quickly with safe and smooth manner.

After the flat face portion 10 on which the endoscopic observation means of the rigid distal end portion 3c of the insertion section 3 of the mother endoscope 1 is introduced into a portion where an examination or treatment should be conducted or specifically, such as a position of a papilla in a duodenum, the daughter endoscope 100 is separated from the mother endoscope 1. As a result, the biopsy channel 13 is restored the state to exhibit its original function as a path for introducing a treatment instrument. At this time, since the instrument riser member 15 is located at the retreated position, the rising operation lever 20 should be operated from the position of the ex-boundary operation mode to the rising operation mode so as to return it within the operation range. As a result, the stopper member 41 returns to engage with the arc groove 40 of the rotating drum 21 by the force of the spring 42 on the stopper member 41, thereby restricting the operation range of the rising operation lever 20.

Therefore, the mother endoscope 1 can be operated as a usual side viewing endoscope, and an appropriate treatment instrument such as a cannulation tube or a stent can be inserted through the biopsy channel 13. The through hole 52 is formed in the front end wall 14c of the instrument rising chamber 14, and the instrument riser member 15 is arranged at a position closer to the proximal side from the through hole 52, and the treatment instrument is guided by the guide surface 15a of the instrument riser member 15 and the direction is changed so that the instrument can be reliably led out of the instrument rising opening 14a. Also, by operating the rising operation lever 20, the treatment instrument led out of the instrument rising opening 14a can be directed to a desired direction. Moreover, by inserting the daughter endoscope 100 into the biopsy channel 13, it is guided by the instrument riser member 15 in the instrument rising chamber 14 and inserted into the papilla so as to conduct an examination of a biliary duct or the like.

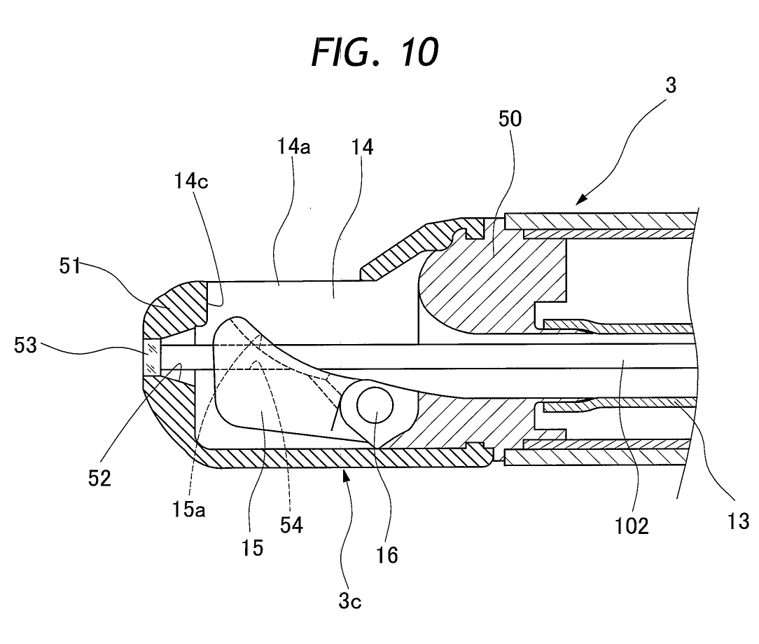

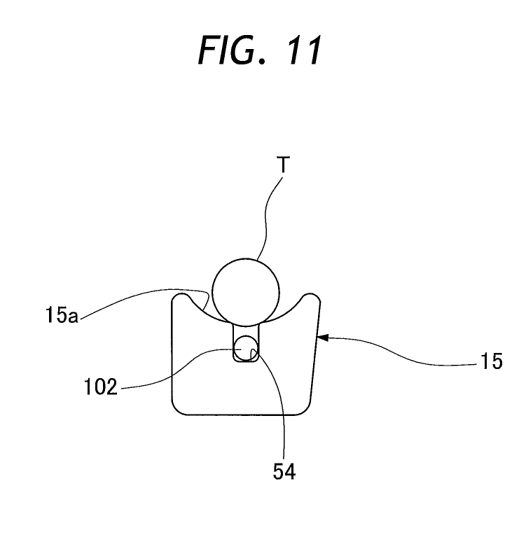

Subsequently, FIGS. 10 and 11 show a second embodiment of the present invention. In this second embodiment, the daughter endoscope passing mechanism disposed in the instrument riser member is constituted by a guide groove 54 as a daughter endoscope passage formed in the guide surface 15a of the instrument riser member 15. This guide groove 54 is to have an open space from the biopsy channel 13 to the perforation window 53 when the instrument riser member 15 is held at the minimum angular position of the operation range in the rising operation mode shown in FIG. 10.

Here, the guide groove 54 has a groove width through which the slender insertion section 102 of the daughter endoscope 100 can pass but smaller than an outer diameter dimension of a general treatment instrument. In a side viewing endoscope, which is the mother endoscope 1, various treatment instruments such as a cannulation tube, a stent or generally-used forceps, a high-frequency treatment instrument and the like are inserted and they all have practically the diameters within a certain range. As indicated by a symbol T in FIG. 11, when the treatment instrument is brought into contact with the instrument riser member 15, the instrument is guided along the guide surface 15a of the instrument riser member 15. When the slender insertion section 102 of the daughter endoscope 100 is brought into contact with the instrument riser member 15, it is introduced in the guide groove 54 and advances forward along the guide groove 54. When the slender insertion section 102 is extended to a position opposing the front end wall 14c in the instrument rising chamber 13, it is brought into contact with the perforation window 53, by which the front visual field is ensured by the daughter endoscope 100.

When the mother endoscope 1 is to be inserted into the body cavity, in order to ensure the forward visual field of the insertion section 3 in the mother endoscope 1, the slender insertion potion 102 of the daughter endoscope 100 is inserted into the treatment instrument introduction portion 5 disposed in the manipulation section 2 in advance. Also, the rising operation lever 20 is not operated and is kept at the position that the instrument riser member 15 is held at the minimum angular position. The distal end of the slender insertion section 102 of the daughter endoscope 100 passes through the guide groove 54 of the instrument riser member 15 and is introduced into the through hole 52 disposed in the front end wall 14c of the instrument rising chamber 14 at a position that the distal end of the slender insertion section 102 is brought into contact with perforation window 53. As a result, the front visual field of the mother endoscope 1 of the side viewing endoscope is ensured by the daughter endoscope 100, and the insertion operation of the insertion section 3 can be performed safely and smoothly, and quickness of the operation is ensured.

In short, the front visual field by the daughter endoscope 100 is required from insertion of the mother endoscope 1 into the body cavity at a predetermined position such as inside the duodenum. After the mother endoscope 1 has been inserted to a portion where an examination or treatment is to be conducted, the daughter endoscope 100 is removed from the biopsy channel 13 so that a required treatment instrument can be inserted in place of the daughter endoscope 100. By providing a shutter to open or close the guide groove 54 as the daughter endoscope passage, the daughter endoscope 100 can be assembled so that it pushes open and passes through the shutter with the distal end portion of its slender insertion section 102 in a state before the mother endoscope 1 is inserted into the body cavity. A construction can be adopted that after the insertion section 3 of the mother endoscope 1 has been inserted to a predetermined position and the daughter endoscope 100 is removed from the biopsy channel 13, the shutter is automatically opened so that the treatment instrument can be inserted smoothly without being blocked.

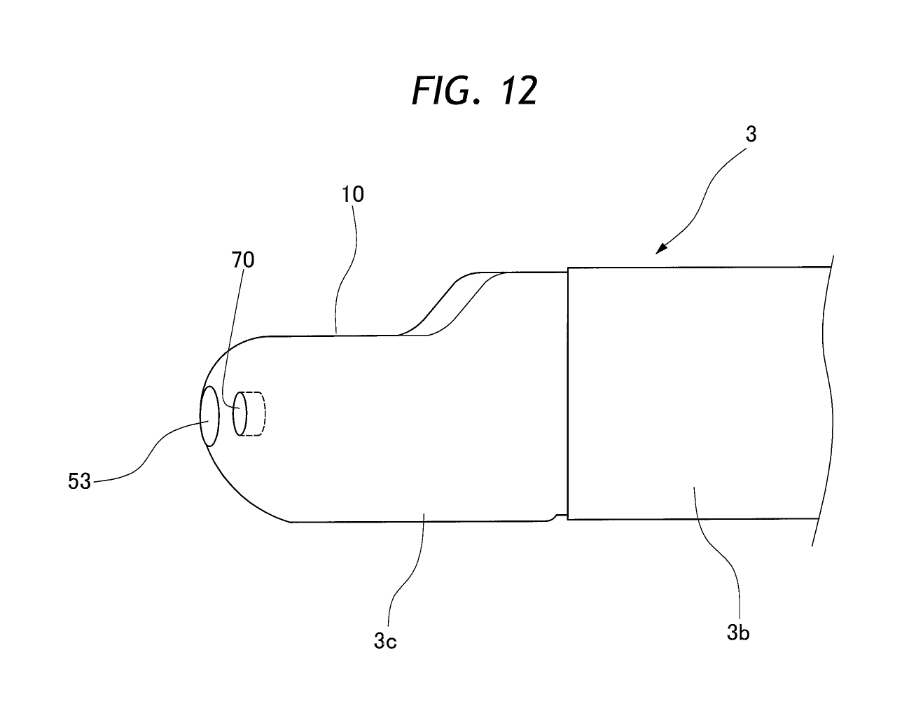

The slender insertion section in the daughter endoscope preferably has as smaller in diameter as possible. For example, as shown in FIG. 12, it may be so configured that a light-emitting diode or the like as a light source 70 is mounted for emitting illumination light forward in the distal end portion of the rigid distal end portion 3c of the insertion section 3 in the mother endoscope 1. The light source 70 can be made to emit light in the body cavity, which eliminates to provide a means for transmitting the illumination light, such as an optical fiber for the daughter endoscope 100, thereby the diameter of the slender insertion section 102 of the daughter endoscope 100 can be further reduced or the volume of the image guides 105 can be increased.

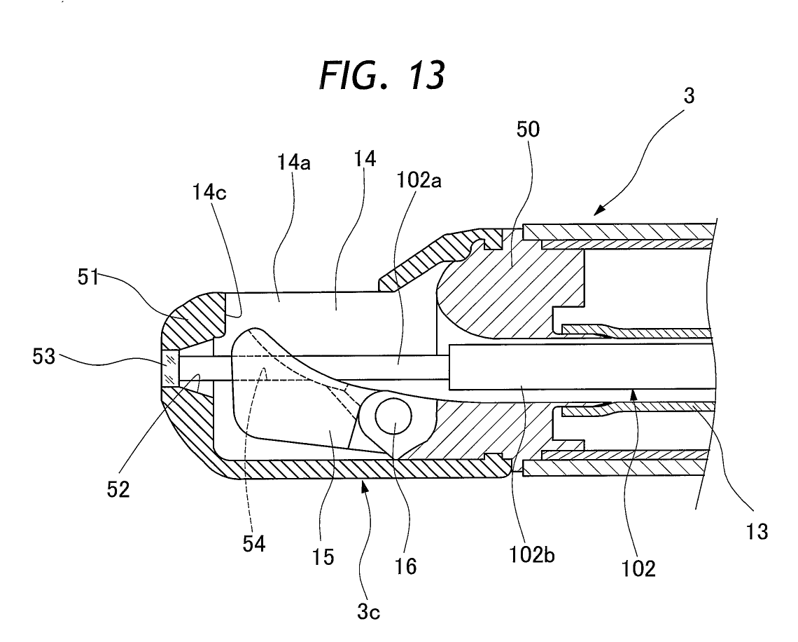

Here, if the diameter of the slender insertion section 102 of the daughter endoscope 100 is reduced, a diameter difference can be provided with respect to the treatment instrument inserted into the biopsy channel 13. However, if the slender insertion section 102 is extremely reduced the diameter, the guidance of the treatment instrument becomes inferior in the biopsy channel 13 toward the instrument rising chamber 14. As a result, the insertion section cannot be introduced into the guide groove 54 formed in the instrument riser member 15 but might be guided along the guide surface 15a toward the direction of the instrument rising opening 14a. Thus, as shown in FIG. 13, a diameter reduced portion 102a in the slender insertion section 102 of the daughter endoscope 100 is limited to a predetermined length on the distal end side, and a portion on the proximal side can be a diameter expanded portion 102b. In this case, the diameter expanded portion 102b has a dimension slightly smaller than the inner diameter of the biopsy channel 13 under a condition that the slender insertion section 102 can smoothly move back and forth in the biopsy channel 13. The diameter reduced portion 102a is given a length substantially equal to an interval from a position where the diameter expanded portion 102b is not brought into contact with the instrument riser member 15 or from the distal end of the biopsy channel 13, for example, to a position where the perforation window 53 is provided if the distal end of the slender insertion section 102 is located at a position opposing the perforation window 53. The diameter expanded portion 102b may be configured such that the diameter of the slender insertion section 102 is expanded or a cylindrical member is attached to a portion corresponding to the diameter expanded portion 102b when the diameter of the slender insertion section 102 has a uniform outer diameter.

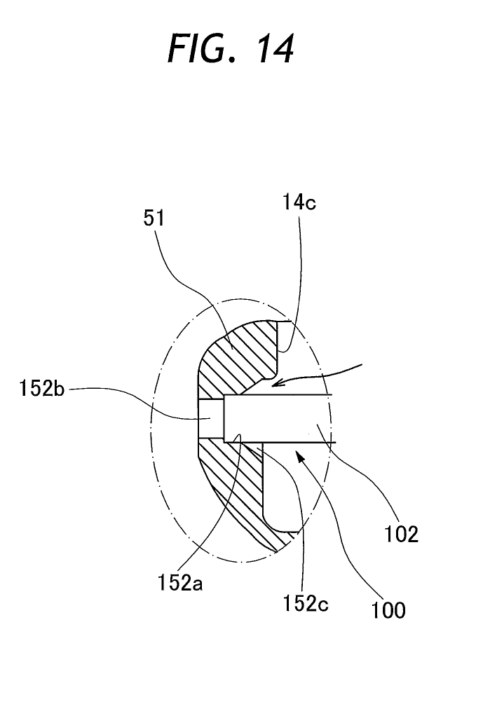

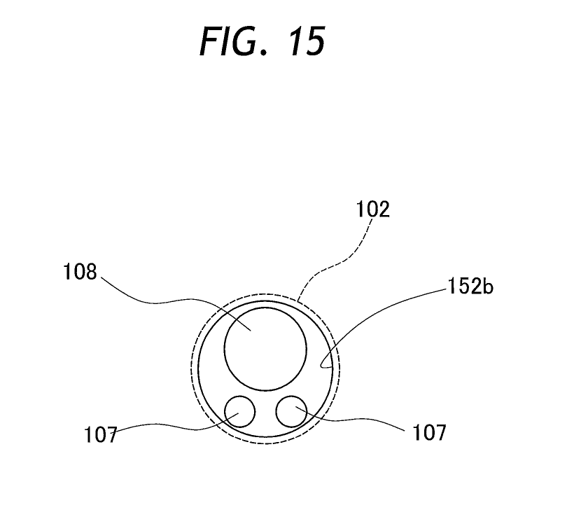

Moreover, in the first embodiment, as the regulating portion for regulating, the slender insertion section 102 of the slender endoscope 100 so that it does not protrude from the through hole made up of the through hole 52 formed in the rigid distal end portion 3c in the insertion section 3 of the mother endoscope 1, the perforation window 53 is provided, but instead of that, a construction as shown in FIG. 14 can be provided. In the construction in FIG. 14, in the rigid distal end portion 3c, in the front end wall 14c located in front of the instrument rising chamber 14, the through hole 152 drilled in the cover member 51 as the through hole has a stepped structure. That is, at the side of the front end wall 14c is a large diameter portion 152a of the through hole 152, while being reduced the diameter of the through hole 152 at the side of the cover member 51 as small diameter portion 152b. The small diameter portion 152b has a inner diameter is smaller than the outer diameter of the slender insertion section 102 of the daughter endoscope 100. Also, a tapered portion 152c is preferably formed on the proximal side of the large diameter portion 152a as necessary. However, in the slender insertion section 102 of the daughter endoscope 100, it is necessary that an illumination lens 107 and an objective lens 108 attached at the distal end should not be covered by the stepped face. Thus, as shown in FIG. 15, a diameter difference between the inner diameter of the slender portion 152b and the outer diameter of the slender insertion section 102 should be extremely small. As a result, a decline in a light amount of the illumination light or limitation on the observation visual field will not occur or can be minimized. If the outer diameter dimension of the slender insertion section 102 is smaller than the inner diameter of the through hole 152, the distal end portion of the slender insertion section 102 may be wrapped by a medical tape or the like so that the slender insertion section 102 does not pass through the through hole 152.

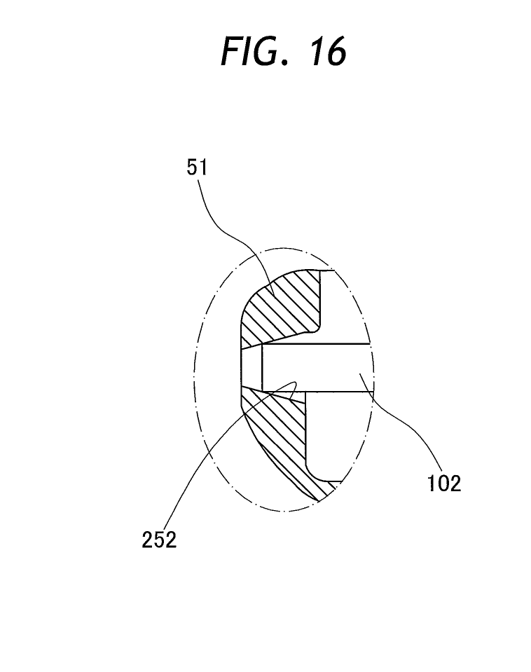

Also, the configuration of the through hole may be a through hole 252 as shown in FIG. 16 such that a taper hole whose diameter is continuously reduced toward proximal side thereof. In this case, the smallest diameter of the through hole 252 should have slightly smaller than the outer diameter of the slender insertion section 102.

1. A side viewing endoscope system comprising:
a mother endoscope of a side viewing type in which an endoscopic observation means having an illumination portion and an observation portion is disposed on a side surface of a rigid distal end portion of an insertion section, a instrument rising chamber installed of a instrument riser member for rising up or sinking down of a treatment instrument is formed in said rigid distal end portion, a biopsy channel provided through an axial direction of said insertion section being communicated with said instrument rising chamber;
a daughter endoscope having a slender insertion section being inserted into said biopsy channel of said mother endoscope and having a viewing field in a forward direction of said distal end of said slender insertion section;
a perforation window foil led at said rigid distal end portion of said mother endoscope and having a viewing field toward the forward direction of said biopsy channel; and
said distal end portion of said daughter endoscope being introduced through said biopsy channel at a position to face said perforation window but not to be protruded from the outer surface of said rigid distal end of said mother endoscope.

2. A side viewing endoscope system according to claim 1, wherein said instrument riser member disposed in said instrument rising chamber is provided with said daughter endoscope passing mechanism to pass said slender insertion section of said daughter endoscope therethrough.

3. A side viewing endoscope system according to claim 2, wherein said instrument riser member is rotatably mounted on a rotational shaft in said rigid distal end portion, a riser operating means is provided on a manipulating section of said mother endoscope for rising up or sinking down said instrument riser member, and said daughter endoscope passing mechanism is adapted to shift said instrument riser member to a retreated position to open said perforation window.

4. A side viewing endoscope system according to claim 3, wherein said riser operating means is adapted to change-over said instrument riser member between a position for a rising operation mode to move said instrument riser member from a minimum angular position to a maximum angular position and a position for an ex-boundary operation mode in which said instrument riser member is displaced to said retreated position, said instrument riser member is normally limited to said rising operation mode, while said riser operating means is shifted to said ex-boundary operation mode beyond the limitation of said rising operation range by means of said riser operating means.

5. A side viewing endoscope system according to claim 2, wherein said daughter endoscope passage is mounted on a surface for guiding said treatment instrument of said instrument riser member toward said biopsy channel.

6. A side viewing endoscope system according to claim 5, wherein said daughter endoscope passage has a guide groove of a width smaller than an outer diameter of said treatment instrument to be inserted into said biopsy channel of said mother endoscope and larger than a diameter of said slender insertion section of said daughter endoscope.

7. A side viewing endoscope system according to claim 1, wherein a through hole is formed in a front end wall of said rigid distal end portion of said mother endoscope to be inserted said slender insertion section thereinto, said through hole has a restricting means to prevent said slender insertion section from protruding out of an outer end surface of said rigid distal end portion.

8. A side viewing endoscope system according to claim 7, wherein said restricting means is constituted by a transparent plate fitted to said through hole.

9. A side viewing endoscope system according to claim 7, wherein said restricting means is formed of a throttle portion with a stepped wall having a diameter smaller than the outer diameter of said slender insertion section.

10. A side viewing endoscope system according to claim 7, wherein said through hole has a taper portion whose diameter is continuously reduced toward the front side, and said restricting means is constituted by the reduced smallest diameter portion of said taper hole which is smaller than the outer diameter of said slender insertion section.

11. A side viewing endoscope system according to any one of claims 1 to 10, wherein said mother endoscope and said daughter endoscope are electronic endoscopes and being displayed a straight view observation image on a monitor screen when said daughter endoscope is inserted into said biopsy channel, while said monitor screen being displayed when said daughter endoscope is removed.

12. A side viewing endoscope system according to claim 2, wherein a through hole is formed in a front end wall of said rigid distal end portion of said mother endoscope to be inserted said slender insertion section thereinto, said through hole has a restricting means to prevent said slender insertion section from protruding out of an outer end surface of said rigid distal end portion.

13. A side viewing endoscope system according to claim 3, wherein a through hole is formed in a front end wall of said rigid distal end portion of said mother endoscope to be inserted said slender insertion section thereinto, said through hole has a restricting means to prevent said slender insertion section from protruding out of an outer end surface of said rigid distal end portion.

14. A side viewing endoscope system according to claim 4, wherein a through hole is formed in a front end wall of said rigid distal end portion of said mother endoscope to be inserted said slender insertion section thereinto, said through hole has a restricting means to prevent said slender insertion section from protruding out of an outer end surface of said rigid distal end portion.

15. A side viewing endoscope system according to claim 5, wherein a through hole is formed in a front end wall of said rigid distal end portion of said mother endoscope to be inserted said slender insertion section thereinto, said through hole has a restricting means to prevent said slender insertion section from protruding out of an outer end surface of said rigid distal end portion.

16. A side viewing endoscope system according to claim 6, wherein a through hole is formed in a front end wall of said rigid distal end portion of said mother endoscope to be inserted said slender insertion section thereinto, said through hole has a restricting means to prevent said slender insertion section from protruding out of an outer end surface of said rigid distal end portion.
